(19) United States
(12) Patent Application Publication
(10) Pub. No.: US 2025/0051702 A1
(43) Pub. Date: Feb. 13, 2025

(54) BIOGAS GENERATION SYSTEM

(71) Applicant: SHIMOSE MICROBES LABORATORY CORPORATION, Shimonoseki-shi (JP)

(72) Inventor: Shinichi SHIMOSE, Shimonoseki-shi (JP)

(21) Appl. No.: 18/930,047

(22) Filed: Oct. 29, 2024

Related U.S. Application Data
(63) Continuation of application No. PCT/JP2023/007147, filed on Feb. 27, 2023.

(30) Foreign Application Priority Data
Apr. 30, 2022 (JP) 2022-075622

(51) Int. Cl. C12M 1/107; B01D 53/26; C12M 1/00; C12M 1/06; C12M 1/34

(52) U.S. Cl. CPC C12M 21/04; B01D 53/265; C12M 27/02; C12M 29/06; C12M 41/12; B01D 2257/80; B01D 2258/05

(57) ABSTRACT

Provided is an energy-saving, environmentally friendly biogas generation system that can reduce the generation of digestive fluid, can efficiently cause methane fermentation by removing ammonia in organic matter regardless of the freshness of the organic matter. The biogas generation system includes a sealed container 2 that contains the organic matter, a stirrer 3 that stirs the organic matter contained in the sealed container 2, a gas-fired boiler 4 that heats the inside of the sealed container 2 to a predetermined temperature, a vacuum pump 5 that reduces pressure in the sealed container 2, and a storage tank 6 that stores biogas generated by methane fermentation of the organic matter in the sealed container 2. Part of the biogas stored in the storage tank 6 is supplied to the gas-fired boiler 4 as fuel.

### TECHNICAL FIELD

The present invention relates to a biogas generation system for generating biogas mainly containing methane by methane fermentation.

### BACKGROUND ART

A system for recovering organic waste such as sewage sludge, human waste, food waste, and livestock waste, or organic matter such as resource crops, or waste thereof by methane fermentation has been devised (see, e.g., Patent Document 1).

A biomass processing system described in Patent Document 1 temporarily stores biogas (methane fermentation gas) generated in a methane fermentation tank in a storage tank (a methane fermentation gas recovery device) before using the biogas for gas power generation. In the biomass processing system described in Patent Document 1, a digestive fluid produced by methane fermentation (a methane fermentation product) is separated into a diffusion liquid (a concentrate) and a dialysate (a desalinated liquid) by a diffusion dialyzer. The diffusion liquid is allowed to pass through a nitrification tank to treat ammonia in the diffusion liquid and through an anammox tank for denitrification, and then is sent to a water treatment facility. This reduces the concentration of organic matter and ammonia in wastewater sent to the water treatment facility, thereby reducing the load on the water treatment facility. The dialysate is returned to the methane fermentation tank to maintain a high concentration of methanogens in the fermentation tank and reduce ammonia which is an inhibitor of the methane fermentation.

### CITATION LIST

### Patent Document

Patent Document 1: Japanese Unexamined Patent Publication No. 2007-44579

### SUMMARY OF THE INVENTION

### Technical Problem

In the biomass processing system of Patent Document 1, a large amount of digestive fluid is generated in the methane fermentation tank. Although attempts have been made to reduce the load on the water treatment facility in terms of components by passing the digestive fluid through the diffusion dialyzer, the nitrification tank, and the anammox tank before sending the digestive fluid to the water treatment facility, the amount of digestive liquid treated in the water treatment facility remains large.

In the system of Patent Document 1, the dialysate is returned to the methane fermentation tank to reduce ammonia in the methane fermentation tank for more efficient methane fermentation. However, this makes the entire system large in scale. The concentration of ammonia in the organic matter fed to the methane fermentation tank varies depending on the freshness of the organic matter. Thus, it is difficult for the system of Patent Document 1 to control the balance between the freshness of the organic matter and the amount of the dialysate returned to the methane fermentation tank.

In view of the foregoing, an object of the present invention is to provide an energy-saving, environmentally friendly biogas generation system that can reduce the generation of digestive fluid, and can achieve efficient methane fermentation regardless of the freshness of organic matter by removing ammonia contained in the organic matter.

### Solution to the Problem

In order to achieve the object, the present invention disclosed in this specification is configured as follows. A first aspect of the present invention is directed to a biogas generation system including: a sealed container that contains organic matter; a stirrer that stirs the organic matter contained in the sealed container; a heater that heats an inside of the sealed container to a predetermined temperature; a pressure reducer that reduces pressure in the sealed container; and a storage tank that stores biogas generated by methane fermentation of the organic matter in the sealed container. Part of the biogas stored in the storage tank is supplied to the heater as fuel.

According to the first aspect, the methane fermentation of the organic matter is caused in the sealed container while stirring the organic matter to generate the biogas. During the methane fermentation, the inside of the sealed container is heated under reduced pressure. This can lower the boiling point of moisture in the organic matter to a temperature at which methanogens are activated, and can maintain the lowered temperature. Activating the methanogens and evaporating the moisture in the organic matter efficiently vaporize ammonia and water contained in the organic matter. Thus, the vapor can be separated from the organic matter containing the methanogens and discharged outside the sealed container. This can reduce ammonia in the organic matter, which is an inhibitor of the methane fermentation, while activating the methanogens, thereby allowing efficient methane fermentation.

In the sealed container used as a methane fermentation tank in the present invention, ammonia can be directly separated from the organic matter unlike the system of Patent Document 1 in which the concentration of ammonia in the organic matter is controlled by returning the dialysate to the methane fermentation tank from the downstream side of the tank. Thus, ammonia in the organic matter can be easily removed regardless of the freshness of the organic matter.

Evaporating the moisture in the organic matter by heating the sealed container can reduce the amount of digestive fluid left after the biogas generation by the methane fermentation of the organic matter more than before. The biogas generated by the methane fermentation is stored in the storage tank, and part of the stored biogas is supplied to the heater as fuel. Thus, no fossil fuel such as heavy oil or coal is required to heat the sealed container. This can make the biogas generation system be energy-saving and environmentally friendly.

A second aspect of the present invention is an embodiment of the first aspect. In the second aspect, the biogas generation system further includes: a condenser that condenses vapor of ammonia and water generated in the sealed container; and a gas-liquid tank that separates ammonia-containing condensed water condensed in the condenser and the biogas. The condenser includes a condenser casing connected to the sealed container by a connecting pipe and having an outlet, the pressure reducer is a vacuum pump, the outlet is connected to the gas-liquid tank by a communication passage, and the vacuum pump is provided at some midpoint of the communication passage.

According to the second aspect, the vapor of ammonia and water generated in the sealed container can be sucked into the condenser casing through the connecting pipe by the drive of the vacuum pump. Further, the vapor can be transformed to ammonia-containing condensed water in the condenser casing. This allows the vapor of ammonia and water generated in the sealed container to be efficiently discharged from the sealed container.

A third aspect of the present invention is an embodiment of the second aspect. In the third aspect, the biogas generation system further includes: a cooling tube that is provided inside the condenser casing and allows cooling water that exchanges heat with the vapor to pass through; and a cooling tower connected to the cooling tube by a cooling water passage and having a water tank for receiving the cooling water. The gas-liquid tank and the water tank are connected by a water supply passage, and the ammonia-containing condensed water stored in the gas-liquid tank is supplied to the water tank through the water supply passage.

According to the third aspect, the ammonia-containing condensed water generated by the condensation of the vapor in the condenser and stored in the gas-liquid tank is sent to the water tank of the cooling tower. The cooling water mixed with the ammonia-containing condensed water circulates between the cooling tube and the cooling tower, and is evaporated little by little and released to the atmosphere. Thus, ammonia is treated in the system.

A fourth aspect of the present invention is an embodiment of the second or third aspect. In the fourth aspect, the biogas generation system further includes: a heat exchanger that cools the biogas stored in the gas-liquid tank to remove water vapor contained in the biogas. The heat exchanger is provided at some midpoint of a biogas supply passage connecting the storage tank and the gas-liquid tank.

According to the fourth aspect, the water vapor contained in the biogas is removed, thereby avoiding troubles such as a malfunction of components due to, for example, freezing of the condensed water in the biogas supply passage in winter.

A fifth aspect of the present invention is an embodiment of the fourth aspect. In the fifth aspect, the biogas generation system further includes: a desulfurizer that desulfurizes the biogas stored in the gas-liquid tank. The desulfurizer is provided at some midpoint of the biogas supply passage between the storage tank and the heat exchanger.

According to the fifth aspect, hydrogen sulfide in the biogas can be removed by the desulfurizer. This can protect the components such as the biogas supply passage and the heater from corrosion by sulfuric acid generated by the reaction of hydrogen sulfide and the condensed water.

A sixth aspect of the present invention is an embodiment of any one of the first to fifth aspects. In the sixth aspect, the heater is a gas-fired boiler that generates heating steam using part of the biogas stored in the storage tank. The biogas generation system further includes: a heating jacket provided on the sealed container; and a heating steam supply passage that connects the gas-fired boiler and the heating jacket. The biogas generation system is configured to send the heating steam generated in the gas-fired boiler to the heating jacket through the heating steam supply passage to heat the inside of the sealed container.

According to the sixth aspect, the gas-fired boiler is driven by the biogas generated by the methane fermentation of the organic matter to heat the sealed container with the heating steam generated in the gas-fired boiler. Thus, the sealed container can be efficiently heated.

A seventh aspect of the invention is an embodiment of any one of the first to sixth aspects. In the seventh aspect, the biogas generation system further includes a biomass boiler that generates steam using, as fuel, a solid product that is produced by methane fermentation of the organic matter in the sealed container and is discharged from a product outlet of the sealed container.

According to the seventh aspect, the inside of the sealed container is heated under reduced pressure for the methane fermentation in the sealed container, and the solid product with low water content is discharged from the product outlet of the sealed container. Use of the biomass boiler allows the solid product to be effectively used as fuel, and the generated steam energy can be used for heating or power generation. Thus, the biogas generation system can be more energy-saving and more environmentally friendly.

An eighth aspect of the present invention is an embodiment of any one of the first to seventh aspects. In the eighth aspect, the biogas generation system further includes: a feeder for feeding the organic matter in the sealed container; and a valve controller that controls the feeder. The feeder includes an inlet, a first feeder valve connected to the bottom of the feeder, a temporary storage connected to the bottom of the first feeder valve, a second feeder valve connected to the bottom of the temporary storage, and a feeder communication pipe connected to the bottom of the second feeder valve and communicating with the inside of the sealed container. The valve controller is configured to open the first feeder valve when the second feeder valve is fully closed, and to open the second feeder valve when the first feeder valve is fully closed so that the organic matter temporarily stored in the temporary storage is fed into the sealed container.

According to the eighth aspect, the first feeder valve and the second feeder valve are sequentially opened and closed, and the organic matter put through the inlet is temporarily stored in the temporary storage and then fed into the sealed container. This can keep the outside of the inlet and the inside of the sealed container from directly communicating with each other. Thus, during the methane fermentation in the sealed container heated under reduced pressure, the organic matter can be continuously fed while maintaining anaerobic conditions in the container. This can achieve efficient methane fermentation.

A ninth aspect of the present invention is an embodiment of the eighth aspect. In the ninth aspect, the first feeder valve and the second feeder valve are gate valves each including a valve housing provided with a through-flow passage and a valve body that moves in a direction perpendicular to the through-flow passage to open and close the through-flow passage.

According to the ninth aspect, it is ensured that the sealed container is airtight and that the whole amount of the organic matter temporarily stored in the temporary storage is fed into the sealed container.

### Advantages of the Invention

The biogas generation system of the present invention can reduce ammonia in the organic matter, which is an inhibitor of the methane fermentation, while activating methanogens, thereby allowing efficient methane fermentation. Ammonia in the organic matter can be easily removed regardless of the freshness of the organic matter. Further, the amount of digestive fluid left after the biogas generation can be greatly reduced. No fossil fuel such as heavy oil or coal is required to heat the sealed container, thereby making the biogas generation system be energy saving and environmentally friendly.

### BRIEF DESCRIPTION OF THE DRAWINGS

FIG. 1 illustrates the overall configuration of a biogas generation system according to a first embodiment of the present invention.

FIG. 2 is a schematic view of the configuration of a main part focusing on a sealed container and a condenser according to the first embodiment.

FIG. 3 illustrates the overall configuration of a biogas generation system according to a second embodiment of the present invention.

FIG. 4 is a schematic view of the configuration of a main part focusing on a sealed container and a condenser according to the second embodiment.

FIG. 5 is a side view of a feeder of the second embodiment.

FIG. 6 is a plan view of a first feeder valve of the second embodiment.

### DESCRIPTION OF EMBODIMENTS

Embodiments of the present invention will be described below with reference to the drawings.

### First Embodiment

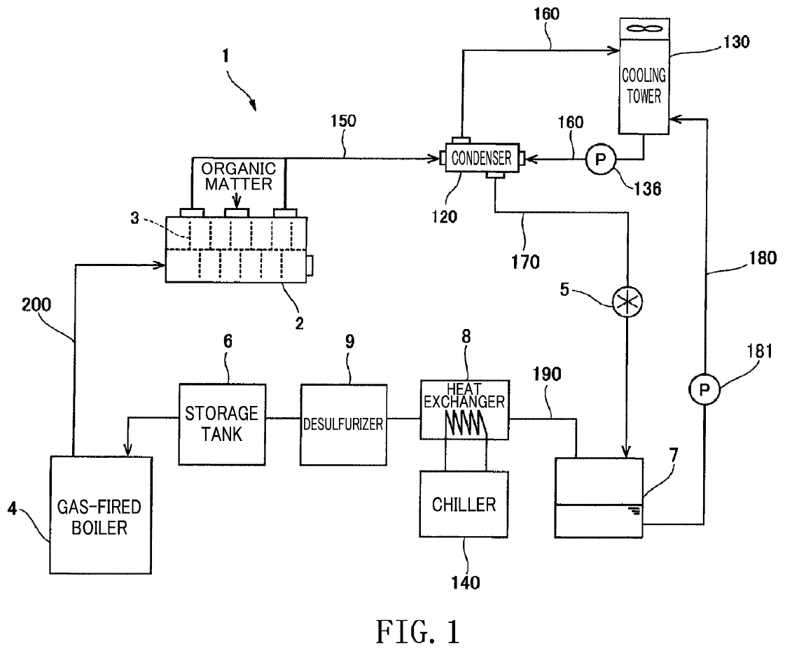

FIG. 1 illustrates the overall configuration of a biogas generation system 1 according to a first embodiment of the present invention. The biogas generation system 1 of the present embodiment includes a sealed container 2 that contains organic matter, a stirrer 3 that stirs the organic matter contained in the sealed container 2, a gas-fired boiler 4 as a heater that heats the inside of the sealed container 2 to a predetermined temperature, a vacuum pump 5 as a pressure reducer that reduces the pressure in the sealed container 2, and a storage tank 6 that stores biogas generated by methane fermentation of the organic matter in the sealed container 2.

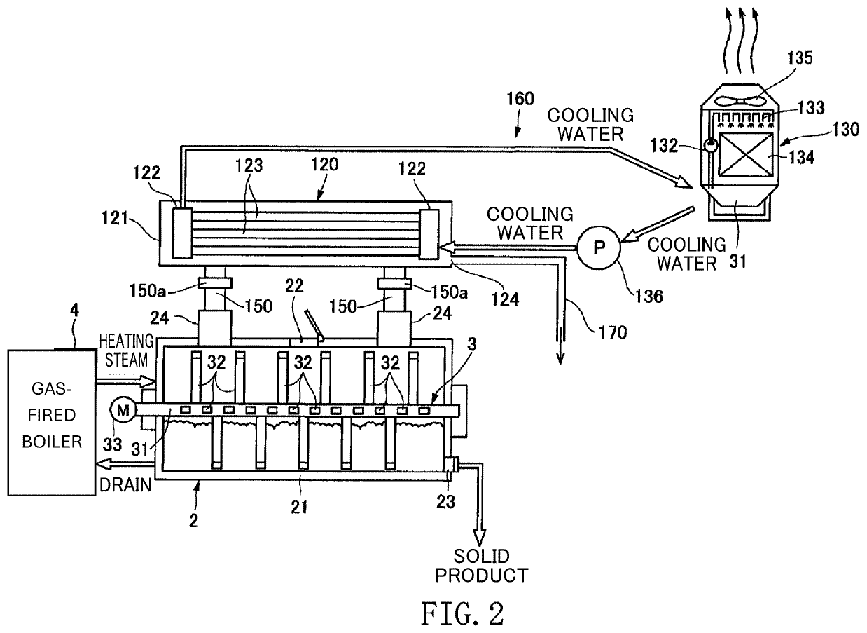

FIG. 2 is a schematic view of the configuration of a main part focusing on the sealed container 2 and a condenser 120 according to the first embodiment. As shown in FIGS. 1 and 2, the sealed container 2 is a substantially cylindrical pressure-resistant tank formed airtight to keep the inside at atmospheric pressure or less. A heating jacket 21 is provided on a peripheral wall of the sealed container 2. The heating jacket 21 is connected to the gas-fired boiler 4 by a heating steam supply passage 200, and heating steam generated in the gas-fired boiler 4 is supplied to the heating jacket 21.

An organic matter inlet 22 is formed at the top of the sealed container 2. The organic matter put into the organic matter inlet 22 includes organic waste such as sewage sludge, human waste, food waste, and livestock waste, or organic matter such as energy crops, industrial crops, or waste thereof. The freshness of the organic matter is not a matter of concern. Two guides 24 protrude from a front portion and rear portion of the top of the sealed container 2. The guides 24 guide biogas and vapor of ammonia and water generated from the heated organic matter to be discharged from the sealed container 2. A product outlet 23 for discharging a solid product left after methane fermentation of the organic matter is formed in the bottom of a rear wall of the sealed container 2.

The stirrer 3 includes a stirring shaft 31 extending in the sealed container 2 in a longitudinal direction of the sealed container 2, multiple stirring plates 32 provided on the stirring shaft 31 to be spaced apart from each other in an axial direction of the stirring shaft 31, and an electric motor 33 that rotates the stirring shaft 31 at a predetermined rotational speed. The organic matter put into the organic matter inlet 22 is heated by the heat of heating steam supplied to the heating jacket 21 while being stirred by the rotation of the stirring shaft 31 and the stirring plates 32. The organic matter, being heated and stirred, moves in the longitudinal direction of the sealed container 2 to be sent to the product outlet 23. As a power source of the electric motor 33, for example, electric power obtained by solar power generation is used. The biogas generation system 1 of the present embodiment is environmentally friendly because other components described later are also configured to use electric power generated without using fossil fuel as much as possible.

One end of a connecting pipe 150 is connected to each of the guides 24 provided for the sealed container 2. The other ends of the connecting pipes 150 are connected to the condenser 120. The condenser 120 condenses the vapor of ammonia and water generated by the methane fermentation in the sealed container 2 into ammonia-containing condensed water. The condenser 120 has a condenser casing 121 extending in a direction parallel to the longitudinal direction of the sealed container 2. The other ends of the connecting pipes 150 are connected to the front and rear portions of the condenser casing 121, respectively. Each of the connecting pipes 150 is provided with an on-off valve 150a. When the methane fermentation of the organic matter in the sealed container 2 is stopped, the on-off valves 150a are closed to keep the air in the sealed container 2 from being sucked into the condenser casing 121.

A plurality of cooling tubes 123 through which cooling water passes is provided in the condenser casing 121. The cooling water passing through the cooling tubes 123 exchanges heat with the vapor of water and ammonia in the condenser 120. The cooling tubes 123 are supported by a pair of heads 122 inside the condenser casing 121.

The cooling tubes 123 are connected to a cooling tower 130 via a cooling water passage 160. The cooling tower 130 includes a water tank 131 for receiving the cooling water. The cooling water flowed through the cooling tubes 123 in the condenser 120 and increased in temperature due to heat exchange with the high-temperature vapor flows into the water tank 131 of the cooling tower 130 through the cooling water passage 160, as schematically indicated by open arrows in FIG. 2.

The cooling tower 130 includes a pump 132 that pumps up the cooling water from the water tank 131, a nozzle 133 that sprays the pumped cooling water, a downflow portion 134, and a fan 135. The cooling water pumped up by the pump 132 and sprayed from the nozzle 133 is lowered in temperature by the air blowing from the fan 135 while flowing down in the downflow portion 134. The cooling water flows down through the downflow portion 134, and then flows into the water tank 131 again.

The cooling water cooled by the cooling tower 130 is sent to the condenser 120 by a cooling water pump 136 through the cooling water passage 160. The cooling water sent to the condenser 120 flows through the plurality of cooling tubes 123 again, and is increased in temperature by the heat exchange with the vapor of water and ammonia generated in the sealed container 2 as described above. Then, the cooling water with the increased temperature passes through the cooling water passage 160 again, and flows into the water tank 131 of the cooling tower 130. That is, the cooling water circulates between the condenser 120 and the cooling tower 130 through the cooling water passage 160.

The condenser casing 121 is provided with an outlet 124. The ammonia-containing condensed water produced by the condensation of the vapor of ammonia and water and the biogas containing methane and carbon dioxide as main ingredients are discharged outside the condenser casing 121 from the outlet 124. The outlet 124 is connected to a gas-liquid tank 7 by a communication passage 170. In the gas-liquid tank 7, the mixture of the ammonia-containing condensed water and the biogas supplied through the communication passage 170 is separated into the ammonia-containing condensed water and the biogas.

As shown in FIG. 1, a vacuum pump 5 is provided at some midpoint of the communication passage 170. The vacuum pump 5 is, for example, a water-sealed vacuum pump 5. Although not shown, an eccentric impeller is rotated in a cylindrical casing of the vacuum pump 5 containing water to press the water against an inner cylindrical wall of the casing by a centrifugal force, causing a water ring. The vacuum pump 5 generates a negative pressure using an air chamber formed inside the water ring.

The vacuum pump 5 is connected to the condenser 120 via the communication passage 170, and the condenser 120 is connected to the sealed container 2 via the connecting pipes 150. Thus, the inside of the sealed container 2 is reduced in pressure by the operation of the vacuum pump 5. In the sealed container 2, the methane fermentation of the organic matter occurs to generate the biogas containing methane and carbon dioxide as the main ingredients and the vapor of ammonia and water. When the sealed container 2 is reduced in pressure, the biogas and the vapor of ammonia and water in the sealed container 2 move to the condenser 120 through the connecting pipes 150. The biogas and the vapor of ammonia and water turn to the biogas and the ammonia-containing condensed water in the condenser 120. Further, the biogas and the ammonia-containing condensed water are transferred from the outlet 124 of the condenser 120 to the gas-liquid tank 7 through the communication passage 170 by the operation of the vacuum pump 5.

The gas-liquid tank 7 stores the biogas in an upper space and the ammonia-containing condensed water in a lower space, thereby separating the biogas which is gas and the ammonia-containing condensed water which is liquid from each other. The upper space in the gas-liquid tank 7 and the gas-fired boiler 4 are connected by a biogas supply passage 190. A heat exchanger 8, a desulfurizer 9, and a storage tank 6 are provided along the biogas supply passage 190 in this order from the gas-liquid tank 7.

The lower space in the gas-liquid tank 7 is connected to the water tank 131 of the cooling tower 130 via the water supply passage 180. A water supply pump 181 is provided at some midpoint of the water supply passage 180. When the water supply pump 181 is operated, the ammonia-containing condensed water stored in the gas-liquid tank 7 is supplied to the water tank 131 through the water supply passage 180. The water supply passage 180 is designed to keep the biogas from being released to the atmosphere from the gas-liquid tank 7 via the cooling tower 130 when the gas-liquid tank 7 runs out of the ammonia-containing condensed water. For example, a pipe of the water supply passage 180 includes a bent portion for leaving water at all times so that the pipe is divided into an upstream side and a downstream side by the water left in the bent portion.

The heat exchanger 8 is provided to remove water vapor contained in the biogas by cooling the biogas supplied from the gas-liquid tank 7 through the biogas supply passage 190. That is, although most of the water vapor is condensed into condensed water in the condenser 120, some of the water vapor remains without being condensed. Thus, the water vapor in the biogas which is not condensed and remains is removed in the heat exchanger 8. Specifically, the biogas is cooled by a chiller 140 attached to the heat exchanger 8 to remove the water vapor from the biogas.

The desulfurizer 9 is a device for desulfurizing the biogas supplied from the gas-liquid tank 7 through the biogas supply passage 190. Specifically, the desulfurizer 9 is provided to remove hydrogen sulfide contained in a trace amount in the biogas. If not removed, hydrogen sulfide reacts with the condensed water to produce sulfuric acid. Sulfuric acid thus produced may corrode the pipes and components provided along the biogas supply passage 190 and the gas-fired boiler 4 connected to the downstream end of the biogas supply passage 190. The desulfurizer 9 avoids the corrosion and the accompanying troubles such as a malfunction of the components.

The storage tank 6 stores the biogas sent from the gas-liquid tank 7 through the heat exchanger 8 and the desulfurizer 9. The biogas stored in the storage tank 6 contains methane and carbon dioxide as the main ingredients. The concentration of methane contained in the biogas in the storage tank 6 is 60% or more. Thus, the biogas stored in the storage tank 6 has a methane concentration suitable for use as fuel. The biogas stored in the storage tank 6 is also used in another system as a product of the biogas generation system 1. As a feature of the biogas generation system 1 of the present embodiment, part of the biogas stored in the storage tank 6 is supplied to the gas-fired boiler 4 as fuel.

The gas-fired boiler 4 is constituted of, for example, a gas-fired once-through boiler, and generates heating steam. The heating steam generated by the gas-fired boiler 4 is sent to the heating jacket 21 through the heating steam supply passage 200 to heat the inside of the sealed container 2.

The operation of the biogas generation system 1 configured as described above will be described with reference to FIGS. 1 and 2. First, the organic matter fed into the sealed container 2 through the organic matter inlet 22 is heated by the heating jacket 21 heated by the heating steam from the gas-fired boiler 4. At the same time, the pressure in the sealed container 2 is reduced by the drive of the vacuum pump 5. The pressure reduction lowers the boiling point of water. Thus, the inside of the sealed container 2 is maintained at 50° C. to 60° C., and moisture contained in the organic matter is boiled at this temperature. The organic matter is stirred by the stirring plates 32 of the stirrer 3. In this situation, the methane fermentation of the organic matter is promoted, and the biogas containing methane and carbon dioxide as main ingredients and the vapor of water and ammonia are generated from the organic matter. The generated biogas and vapor are transferred to the condenser 120 through the connecting pipes 150 by the action of the vacuum pump 5. The biogas and the vapor are continuously transferred. Thus, when the biogas and the vapor are generated from the organic matter and transferred to the condenser 120 through the connecting pipes 150, the biogas and the vapor that are previously generated and transferred to the condenser 120 through the connecting pipes 150 do not serve as a resistance. Further, when the vapor of ammonia and water is generated from the organic matter and transferred from the sealed container 2 to the condenser 120, ammonia in the organic matter, which is an inhibitor of the methane fermentation, is reduced, thereby maintaining a high concentration of methanogens.

In the condenser 120, the biogas and the vapor of ammonia and water exchange heat with the cooling tubes 123, and the vapor turns to ammonia-containing condensed water. The biogas and the ammonia-containing condensed water pass through the communication passage 170 and are stored in the gas-liquid tank 7. In the gas-liquid tank 7, the ammonia-containing condensed water is sent to the water tank 131 of the cooling tower 130 through the water supply passage 180. In the cooling tower 130, the cooling water originally present and the ammonia-containing condensed water sent through the water supply passage 180 are mixed together, and the mixture circulates between the condenser 120 and the cooling tower 130 through the cooling water passage 160. During the circulation, the cooling water containing ammonia is evaporated little by little, releasing ammonia to the atmosphere from the cooling tower 130. Thus, the ammonia is treated. The ammonia condensed water is also mixed into sealing water in the water-sealed vacuum pump 5. Thus, a sealing water tank (not shown) for supplying the sealing water to the vacuum pump 5 may be provided with a mechanism for releasing ammonia to the atmosphere, or a pipe for the sealing water may be connected to the water tank 131 of the cooling tower 130.

The biogas in the gas-liquid tank 7 is sent to the storage tank 6 through the biogas supply passage 190. While the biogas is moving from the gas-liquid tank 7 to the storage tank 6, the heat exchanger 8 removes water vapor remaining in the biogas, and the desulfurizer 9 removes hydrogen sulfide in the biogas. The storage tank 6 stores the biogas having a methane concentration of 60% or more, and part of the biogas is burned as fuel in the gas-fired boiler 4. Carbon dioxide contained in the biogas is discharged from a stack (not shown) of the gas-fired boiler 4 in the end. However, this carbon dioxide is not produced by burning fossil fuel, achieving carbon neutrality.

As the methane fermentation progresses, the organic matter in the sealed container 2 is condensed and dried with the generation of the biogas and the vapor. Finally, the organic matter is largely reduced from the initial amount, and a solid product having fiber contents is left. The remaining solid product is discharged from the product outlet 23.

As described above, the biogas generation system 1 of the present embodiment includes the sealed container 2 that contains the organic matter, the stirrer 3 that stirs the organic matter contained in the sealed container 2, the gas-fired boiler 4 as a heater that heats the inside of the sealed container 2 to a predetermined temperature, the vacuum pump 5 that reduces the pressure in the sealed container 2, and the storage tank 6 that stores the biogas generated by the methane fermentation of the organic matter in the sealed container 2. Part of the biogas stored in the storage tank 6 is supplied to the gas-fired boiler 4 as fuel.

This configuration allows the methane fermentation of the organic matter while stirring the organic matter with the stirrer 3 in the sealed container 2, thereby generating the biogas. During the methane fermentation, the inside of the sealed container 2 is heated under reduced pressure. This can lower the boiling point of moisture in the organic matter to a temperature at which the methanogens are activated, for example, 50° C. to 60° C., and can maintain the lowered temperature. Activating the methanogens and evaporating the moisture in the organic matter efficiently vaporize ammonia and water contained in the organic matter.

Thus, the vapor can be separated from the organic matter containing the methanogens and discharged outside the sealed container 2. This can reduce ammonia in the organic matter, which is an inhibitor of the methane fermentation, while activating the methanogens, thereby allowing efficient methane fermentation. In the sealed container 2, ammonia can be directly separated from the organic matter. Thus, ammonia in the organic matter can be easily removed regardless of the freshness of the organic matter.

Evaporating the moisture in the organic matter by heating the sealed container 2 can reduce the amount of digestive fluid left after the biogas generation by the methane fermentation of the organic matter more than before. The biogas generated by the methane fermentation is stored in the storage tank 6, and part of the stored biogas is supplied to the gas-fired boiler 4 as fuel. Thus, no fossil fuel such as heavy oil or coal is required to heat the sealed container 2. Thus, the biogas generation system 1 can be energy-saving and environmentally friendly.

In the present embodiment, the biogas generation system includes the condenser 120 that condenses the vapor of ammonia and water generated in the sealed container 2 and the gas-liquid tank 7 that separates the ammonia-containing condensed water condensed in the condenser 120 and the biogas. The condenser 120 includes a condenser casing 121 connected to the sealed container 2 by the connecting pipe 150 and having the outlet 124. The outlet 124 is connected to the gas-liquid tank 7 by the communication passage 170, and the vacuum pump 5 is provided at some midpoint of the communication passage 170.

This configuration allows the vapor of ammonia and water generated in the sealed container 2 to be sucked into the condenser casing 121 through the connecting pipes 150 by the drive of the vacuum pump 5. Further, the vapor can be transformed to the ammonia-containing condensed water in the condenser casing 121. This allows the vapor of ammonia and water generated in the sealed container 2 to be efficiently discharged from the sealed container 2.

In the present embodiment, the biogas generation system further includes the cooling tubes 123 that are provided inside the condenser casing 121 and allow the cooling water for heat exchange with the vapor to pass through and the cooling tower 130 connected to the cooling tubes 123 by the cooling water passage 160 and having the water tank 131 for receiving the cooling water. The gas-liquid tank 7 and the water tank 131 are connected by the water supply passage 180, and the ammonia-containing condensed water stored in the gas-liquid tank 7 is supplied to the water tank 131 through the water supply passage 180.

This configuration allows the ammonia-containing condensed water generated by the condensation of the vapor in the condenser 120 and stored in the gas-liquid tank 7 to be sent to the water tank 131 of the cooling tower 130. The cooling water mixed with the ammonia-containing condensed water is evaporated little by little and released to the atmosphere while circulating between the cooling tubes 123 and the cooling tower 130. Thus, ammonia is treated in the system.

In the present embodiment, the biogas generation system further includes the heat exchanger 8 that cools the biogas stored in the gas-liquid tank 7 to remove water vapor contained in the biogas. The heat exchanger 8 is provided at some midpoint of the biogas supply passage 190 connecting the storage tank 6 and the gas-liquid tank 7. This configuration allows removal of the water vapor contained in the biogas, thereby avoiding troubles such as a malfunction of components due to, for example, freezing of the condensed water in the biogas supply passage 190 in winter.

In the present embodiment, the biogas generation system includes the desulfurizer 9 that desulfurizes the biogas stored in the gas-liquid tank 7. The desulfurizer 9 is provided at some midpoint of the biogas supply passage 190 between the storage tank 6 and the heat exchanger 8. This configuration allows removal of hydrogen sulfide in the biogas by the desulfurizer 9. This can protect the components such as the biogas supply passage 190 and the gas-fired boiler 4 from corrosion by sulfuric acid generated by the reaction of hydrogen sulfide and the condensed water.

In the present embodiment, the gas-fired boiler 4 generates heating steam using part of the biogas stored in the storage tank 6 as fuel. The biogas generation system further includes the heating jacket 21 provided on the sealed container 2 and the heating steam supply passage 200 that connects the gas-fired boiler 4 and the heating jacket 21. The biogas generation system is configured to send the heating steam generated in the gas-fired boiler 4 to the heating jacket 21 through the heating steam supply passage 200 to heat the inside of the sealed container 2. In this configuration, the gas-fired boiler 4 is driven by the biogas generated by the methane fermentation of the organic matter to heat the sealed container 2 with the heating steam generated in the gas-fired boiler 4. Thus, the sealed container 2 can be efficiently heated.

### Second Embodiment

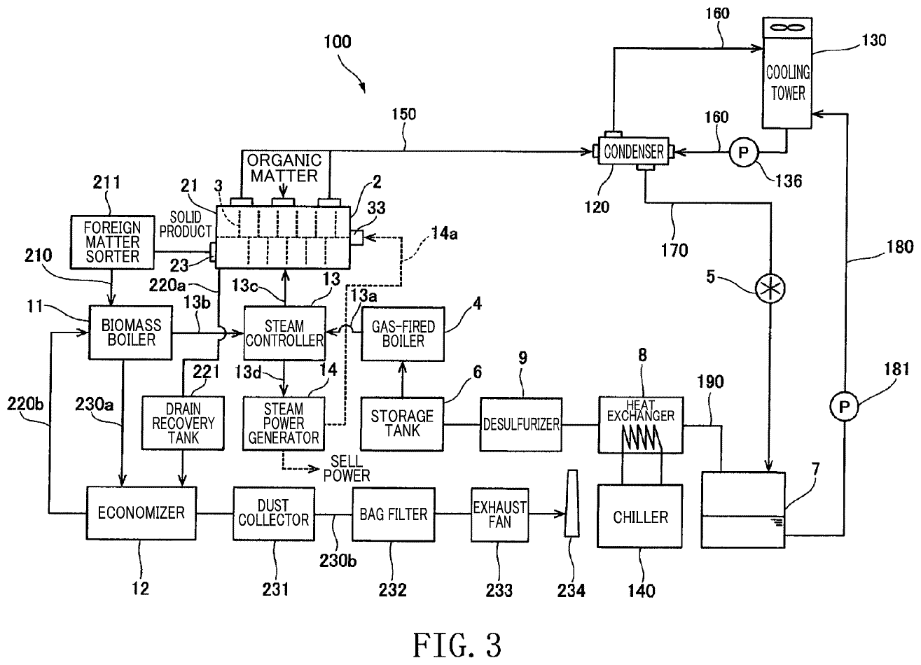

FIG. 3 illustrates the overall configuration of a biogas generation system 100 according to a second embodiment of the present invention. In the following description of the second embodiment, the same components as those of the first embodiment are denoted by the same reference numerals, and the description thereof will be omitted. The biogas generation system 100 of the second embodiment includes a biomass boiler 11. The biomass boiler 11 generates steam using, as fuel, a solid product generated by methane fermentation of organic matter in a sealed container 2 and discharged from a product outlet 23 of the sealed container 2. The biomass boiler 11 is a gas fuel boiler. The gas fuel boiler burns the solid product to generate combustion gas such as carbon monoxide gas, hydrogen gas, and methane gas from the solid product. The gas is burned to generate heat, and the heat turns water to steam.

The biomass boiler 11 is connected to the product outlet 23 of the sealed container 2 by a solid product supply passage 210. A foreign matter sorter 211 is provided at some midpoint of the solid product supply passage 210. The foreign matter sorter 211 removes substances such as metals unsuitable as the fuel for the biomass boiler 11. Exhaust gas generated by the biomass boiler 11 passes through a first exhaust passage 230a and a second exhaust passage 230b, and is released to the outside air from a stack 234. An economizer 12 is provided between the first exhaust passage 230a and the second exhaust passage 230b. A dust collector 231, a bag filter 232, and an exhaust fan 233 are provided along the second exhaust passage 230b downstream of the economizer 12. Dust in the exhaust gas is removed by passing the exhaust gas through the dust collector 231 and the bag filter 232.

One end of a first drain passage 220a is connected to the sealed container 2. The other end of the first drain passage 220a is connected to the economizer 12. A drain recovery tank 221 is provided at some midpoint of the first drain passage 220a. The drain recovery tank 221 recovers water accumulated at the bottom of the sealed container 2 or the heating jacket 21 during the methane fermentation in the sealed container 2. The water temporarily stored in the drain recovery tank 221 is appropriately sent to the economizer 12, and is heated to be hot water by the heat of the exhaust gas entering the economizer 12 through the first exhaust passage 230a. The hot water is supplied from the economizer 12 to the biomass boiler 11 through the second drain passage 220b.

The biogas generation system 100 of the second embodiment includes a steam controller 13 that controls the amount of steam generated by a gas-fired boiler 4 and the biomass boiler 11. The steam controller 13 and the gas-fired boiler 4 are connected by a first steam passage 13a. The steam controller 13 and the biomass boiler 11 are connected by a second steam passage 13b. The steam controller 13 and the heating jacket 21 are connected by a third steam passage 13c. The steam controller 13 supplies the steam generated in the gas-fired boiler 4 and the biomass boiler 11 to the heating jacket 21. The steam controller 13 is connected to a steam power generator 14 by a fourth steam passage 13d. The steam controller 13 also supplies the steam generated in the gas-fired boiler 4 and the biomass boiler 11 to the steam power generator 14. The steam power generator 14 is connected to an electric motor 33 of a stirrer 3 by a power supply passage 14a. The electric power generated by the steam power generator 14 is supplied to the electric motor 33 through the power supply passage 14a and is used as rotational driving energy of the stirring shaft 31. A surplus of the electric power generated by the steam power generator 14 is sold.

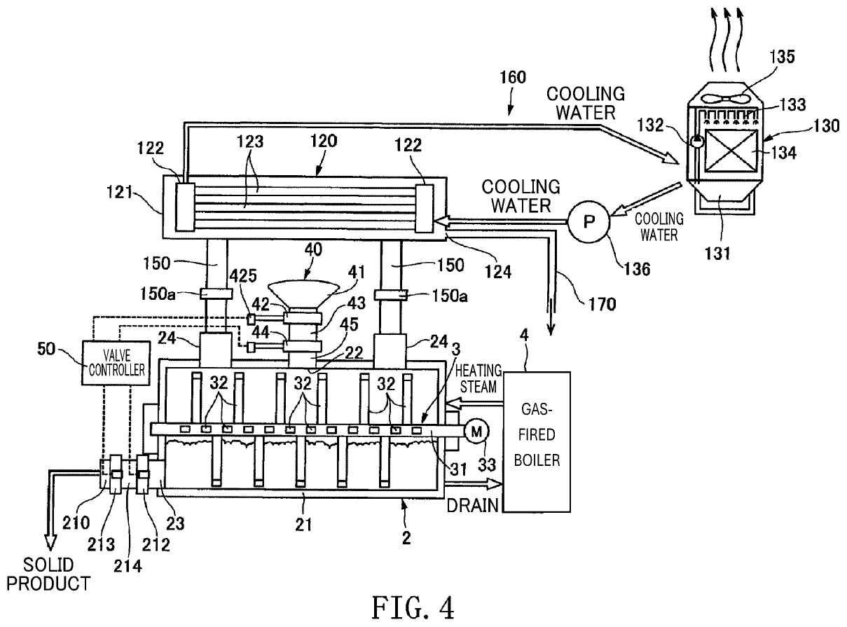

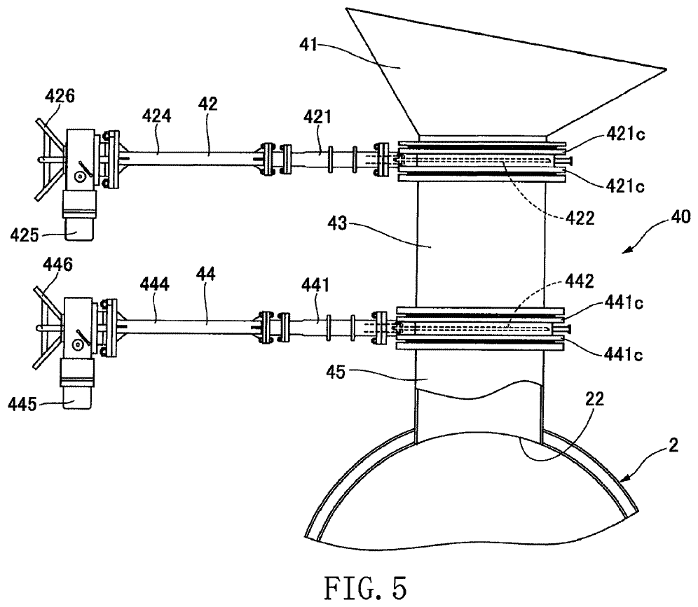

FIG. 4 is a schematic vies of the configuration of a main part focusing on the sealed container 2 and the condenser 120 of the second embodiment. FIG. 5 is a side view of a feeder 40 of the second embodiment. As shown in FIGS. 4 and 5, in the second embodiment, the feeder 40 is attached to the top of the sealed container 2. The feeder 40 includes an inlet 41, a first feeder valve 42, a temporary storage 43, a second feeder valve 44, and a feeder communication pipe 45.

The inlet 41 is a portion through which the organic matter is put into the sealed container 2 from outside, and is formed as an opening that is flared upward. The first feeder valve 42 is connected to the bottom of the inlet 41. When the organic matter is put into the inlet 41, the first feeder valve 42 is fully opened. The temporary storage 43 is formed in a circular tube shape extending linearly in the vertical direction. The temporary storage 43 is provided to temporarily store the organic matter put into the inlet 41. The second feeder valve 44 is connected to the bottom of the temporary storage 43. When the organic matter is temporarily stored in the temporary storage 43, the second feeder valve 44 is fully closed. The feeder communication pipe 45 is connected to the bottom of the second feeder valve 44. A lower end of the feeder communication pipe 45 is connected to an organic matter inlet 22 formed at the top of the sealed container 2. Thus, the feeder communication pipe 45 communicates with the inside of the sealed container 2.

The feeder 40 is controlled by a valve controller 50. Specifically, the valve controller 50 opens the first feeder valve 42 when the second feeder valve 44 is fully closed. The valve controller 50 opens the second feeder valve 44 when the first feeder valve 42 is fully closed so that the organic matter temporarily stored in the temporary storage 43 is fed into the sealed container 2. That is, the valve controller 50 controls the first feeder valve 42 and the second feeder valve 44 so that the valves do not open at the same time.

As shown in FIG. 4, in the second embodiment, a solid product supply passage 210 is connected to the product outlet 23 of the sealed container 2. A first discharge valve 212 and a second discharge valve 213 are provided in the solid product supply passage 210 near the product outlet 23. A temporary storage 214 is provided between the first discharge valve 212 and the second discharge valve 213. The valve controller 50 controls opening and closing of the first discharge valve 212 and the second discharge valve 213. Specifically, the valve controller 50 opens the first discharge valve 212 when the second discharge valve 213 is fully closed. The valve controller 50 opens the second discharge valve 213 when the first discharge valve 212 is fully closed so that the solid product temporarily stored in the temporary storage 214 is sent to the foreign matter sorter 211. That is, the valve controller 50 controls the first discharge valve 212 and the second discharge valve 213 so that the valves do not open at the same time.

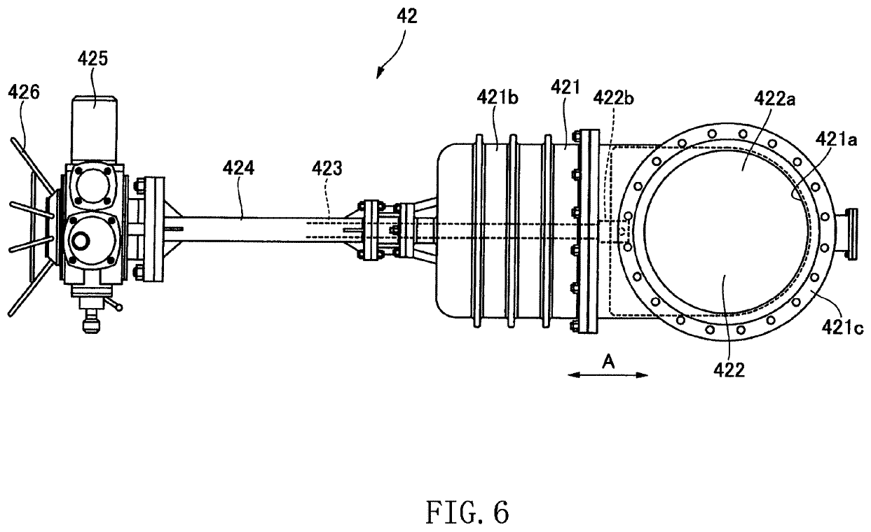

FIG. 6 is a plan view of the first feeder valve 42 of the second embodiment. The first feeder valve 42 and the second feeder valve 44 have the same structure. The first discharge valve 212 and the second discharge valve 213 have the same structure as the first feeder valve 42. The first feeder valve 42 will be taken as a typical example, and its structure will be described in detail below. As shown in FIG. 6, the first feeder valve 42 is a gate valve including a valve housing 421 provided with a through-flow passage 421a and a plate-shaped valve body 422 for opening and closing the through-flow passage 421a. The valve body 422 moves in a direction perpendicular to the direction of extension of the center axis of the through-flow passage 421a to open or close.

The valve housing 421 is elongated in the moving direction of the valve body 422 (the direction A in FIG. 6). The through-flow passage 421a is formed at one end of the valve housing 421 in a longitudinal direction of the valve housing 421. A housing portion 421b for housing the valve body 422 in the open state is formed at the other end of the valve housing 421 in the longitudinal direction. A space inside the housing portion 421b is flat-shaped.

The valve body 422 is provided to move linearly inside the valve housing 421. A cover 422a for blocking the through-flow passage 421a is formed in a portion of the valve body 422 facing the through-flow passage 421a. The cover 422a has a semicircular outer edge portion. A coupling portion 422b for coupling the valve body 422 with a valve rod 423 is provided at an end of the valve body 422 opposite to the cover 422a.

A long cylindrical portion 424 which is long, narrow, and linear in shape is connected to the housing portion 421b of the valve housing 421. A motor 425 and a handle 426 are provided at a distal end of the long cylindrical portion 424. The motor 425 automatically opens and closes the through-flow passage 421a by automatically moving the valve body 422. The handle 426 allows the valve body 422 to move manually so that the through-flow passage 421a is manually opened or closed. The valve rod 423 extends inside the housing portion 421b of the valve housing 421 and the long cylindrical portion 424. One end of the valve rod 423 is connected to the valve body 422 by the coupling portion 422b. The other end of the valve rod 423 is connected to the motor 425 and the handle 426.

The driving force of the motor 425 or the force generated by operating the handle 426 is transmitted to the valve body 422 via the valve rod 423. The drive of the motor 425 or the operation of the handle 426 moves the valve body 422 to open or close. The through-flow passage 421a of the valve housing 421 has a pair of openings with flanges 421c.

As shown in FIG. 5, the first feeder valve 42 is connected to the inlet 41 and the temporary storage 43 at the position of the flanges 421c. The first feeder valve 42 is placed such that the valve body 422 moves horizontally. The long cylindrical portion 424 of the first feeder valve 42 extends horizontally.

The second feeder valve 44 includes a valve housing 441, a through-flow passage (not shown), flanges 441c, a valve body 442, a long cylindrical portion 444, a motor 445, and a handle 446, similarly to the first feeder valve 42. The second feeder valve 44 is connected to the temporary storage 43 and the feeder communication pipe 45 at the position of the flanges 441c. The long cylindrical portion 444 of the second feeder valve 44 extends horizontally.

The motor 425 of the first feeder valve 42 and the motor 445 of the second feeder valve 44 are electrically connected to the valve controller 50. The valve controller 50 controls the motor 425 to open and close the valve body 422 of the first feeder valve 42. The valve controller 50 controls the motor 445 to open and close the valve body 442 of the second feeder valve 44.

As described above, the biogas generation system 100 of the present embodiment includes the biomass boiler 11 that generates steam using the solid product as the fuel. The solid product is produced by the methane fermentation of the organic matter in the sealed container 2 and discharged from the product outlet 23 of the sealed container 2.

According to the above configuration, the inside of the sealed container 2 is heated under reduced pressure for the methane fermentation in the sealed container 2, and the solid product with low water content is discharged from the product outlet 23 of the sealed container 2. Use of the biomass boiler 11 allows the solid product to be effectively used as the fuel, and the generated steam energy can be used for heating or power generation. This makes the system more energy-saving and more environmentally friendly than systems without the biomass boiler 11.

In the present embodiment, the biogas generation system includes the feeder 40 for feeding the organic matter into the sealed container 2 and the valve controller 50 for controlling the feeder 40. The feeder 40 includes the inlet 41, the first feeder valve 42 connected to the bottom of the inlet 41, the temporary storage 43 connected to the bottom of the first feeder valve 42, the second feeder valve 44 connected to the bottom of the temporary storage 43, and the feeder communication pipe 45 connected to the bottom of the second feeder valve 44 and communicating with the inside of the sealed container 2. The valve controller 50 is configured to open the first feeder valve 42 when the second feeder valve 44 is fully closed, and to open the second feeder valve 44 when the first feeder valve 42 is fully closed so that the organic matter temporarily stored in the temporary storage 43 is fed into the sealed container 2.

This configuration can keep the outside of the inlet 41 and the inside of the sealed container 2 from directly communicating with each other. Thus, during the methane fermentation in the sealed container 2 heated under reduced pressure, the organic matter can be continuously fed while maintaining anaerobic conditions in the container. This can achieve efficient methane fermentation.

In the present embodiment, the first discharge valve 212 and the second discharge valve 213 are provided in the solid product supply passage 210 near the product outlet 23 of the sealed container 2. A temporary storage 214 is provided between the first discharge valve 212 and the second discharge valve 213. The valve controller 50 opens the first discharge valve 212 when the second discharge valve 213 is fully closed. The valve controller 50 is configured to open the second discharge valve 213 when the first discharge valve 212 is fully closed so that the solid product temporarily stored in the temporary storage 214 is sent to the foreign matter sorter 211.

This configuration can keep the foreign matter sorter 211 and the inside of the sealed container 2 from directly communicating with each other. Thus, during the methane fermentation in the sealed container 2 heated under reduced pressure, the solid product can be continuously discharged while maintaining anaerobic conditions in the container. This can achieve efficient methane fermentation.

In the present embodiment, the first feeder valve 42, the second feeder valve 44, the first discharge valve 212, and the second discharge valve 213 are gate valves. This configuration can ensure the airtightness of the sealed container 2, the feeding of the organic matter, and the discharge of the solid product.

The disclosed embodiments are illustrative in all respects and do not serve as a basis for restrictive interpretation. The scope of the present invention is not interpreted only by the above-described embodiments, but is defined based on the description of the claims. The scope of the present invention includes all modifications within the meaning and scope equivalent to the scope of the claims.

For example, the biogas generation system 100 of the second embodiment includes the two feeder valves, namely, the first and second feeder valves 42 and 44, and the two discharge valves, namely, the first and second discharge valves 212 and 213. The present invention is not limited to this example, and the system may have a single feeder valve and/or a single discharge valve.

In the second embodiment, the gate valves are used as the first feeder valve 42, the second feeder valve 44, the first discharge valve 212, and the second discharge valve 213. The present invention is not limited to this example, and other valves than the gate valve may be used.

In the second embodiment, the temporary storage 43 of the feeder 40 is formed of a circular pipe extending linearly in the vertical direction. The present invention is not limited to this example, and the temporary storage 43 may have a center portion greatly expanded in the horizontal direction or any other suitable shape so that a larger amount of organic matter can be temporarily stored.

### INDUSTRIAL APPLICABILITY

The present invention is applicable to a biogas generation system that generates biogas mainly containing methane by methane fermentation.

### DESCRIPTION OF REFERENCE CHARACTERS

1, 100 Biogas Generation System
2 Sealed Container
3 Stirrer
4 Gas-Fired Boiler (Heater)
5 Vacuum Pump (Pressure Reducer)
6 Storage Tank
7 Gas-Liquid Tank
8 Heat Exchanger
9 Desulfurizer
21 Heating Jacket
23 Product Outlet
40 Feeder
41 Inlet
42 First Feeder Valve
43 Temporary Storage
44 Second Feeder Valve
45 Feeder Communication Pipe
50 Valve Controller
120 Condenser
121 Condenser Casing
123 Cooling Tube
124 Outlet
130 Cooling Tower
131 Water Tank
150 Connecting Pipe
160 Cooling Water Passage
170 Communication Passage
180 Water Supply Passage
190 Biogas Supply Passage
200 Heating Steam Supply Passage
421, 441 Valve Housing
422, 442 Valve Body

1. A biogas generation system comprising:
a sealed container that contains organic matter;
a stirrer that stirs the organic matter contained in the sealed container;
a heater that heats an inside of the sealed container to a predetermined temperature;
a pressure reducer that reduces pressure in the sealed container; and
a storage tank that stores biogas generated by methane fermentation of the organic matter in the sealed container, wherein
part of the biogas stored in the storage tank is supplied to the heater as fuel.

2. The biogas generation system of claim 1, further comprising:
a condenser that condenses vapor of ammonia and water generated in the sealed container; and
a gas-liquid tank that separates ammonia-containing condensed water condensed in the condenser and the biogas, wherein
the condenser includes a condenser casing connected to the sealed container by a connecting pipe and having an outlet,
the pressure reducer is a vacuum pump,
the outlet is connected to the gas-liquid tank by a communication passage, and
the vacuum pump is provided at some midpoint of the communication passage.

3. The biogas generation system of claim 2, further comprising:
a cooling tube that is provided inside the condenser casing and allows cooling water that exchanges heat with the vapor to pass through; and
a cooling tower connected to the cooling tube by a cooling water passage and having a water tank for receiving the cooling water, wherein
the gas-liquid tank and the water tank are connected by a water supply passage, and
the ammonia-containing condensed water stored in the gas-liquid tank is supplied to the water tank through the water supply passage.

4. The biogas generation system of claim 2, further comprising:
a heat exchanger that cools the biogas stored in the gas-liquid tank to remove water vapor contained in the biogas, wherein
the heat exchanger is provided at some midpoint of a biogas supply passage connecting the storage tank and the gas-liquid tank.

5. The biogas generation system of claim 4, further comprising:
a desulfurizer that desulfurizes the biogas stored in the gas-liquid tank, wherein
the desulfurizer is provided at some midpoint of the biogas supply passage between the storage tank and the heat exchanger.

6. The biogas generation system of claim 1, wherein
the heater is a gas-fired boiler that generates heating steam using part of the biogas stored in the storage tank, and
the biogas generation system further comprises:
a heating jacket provided on the sealed container; and
a heating steam supply passage that connects the gas-fired boiler and the heating jacket, and
the biogas generation system is configured to send the heating steam generated in the gas-fired boiler to the heating jacket through the heating steam supply passage to heat the inside of the sealed container.

7. The biogas generation system of claim 1, further comprising:
a biomass boiler that generates steam using, as fuel, a solid product that is produced by methane fermentation of the organic matter in the sealed container and is discharged from a product outlet of the sealed container.

8. The biogas generation system of claim 1, further comprising:
a feeder for feeding the organic matter in the sealed container; and
a valve controller that controls the feeder, wherein
the feeder includes an inlet, a first feeder valve connected to a bottom of the feeder, a temporary storage connected to a bottom of the first feeder valve, a second feeder valve connected to a bottom of the temporary storage, and a feeder communication pipe connected to a bottom of the second feeder valve and communicating with an inside of the sealed container, and
the valve controller is configured to open the first feeder valve when the second feeder valve is fully closed, and to open the second feeder valve when the first feeder valve is fully closed so that the organic matter temporarily stored in the temporary storage is fed into the sealed container.

9. The biogas generation system of claim 8, wherein
the first feeder valve and the second feeder valve are gate valves each including a valve housing provided with a through-flow passage and a valve body that moves in a direction perpendicular to the through-flow passage to open and close the through-flow passage.
